(19) United States
(12) Patent Application Publication
(10) Pub. No.: US 2024/0003792 A1
(43) Pub. Date: Jan. 4, 2024

(54) METHOD FOR PREPARING CELL-DERIVED VESICLE AND USE THEREOF

(71) Applicant: MDimune Inc., Seoul (KR)

(72) Inventors: Shin Gyu Bae, Gyeonggi-do (KR); Seung Wook Oh, Gyeonggi-do (KR); Dong Woo Han, Gyeonggi-do (KR)

(21) Appl. No.: 17/928,177

(22) PCT Filed: Apr. 19, 2021
(86) PCT No.: PCT/KR2021/004884

§ 371 Date: Nov. 28, 2022

(30) Foreign Application Priority Data
May 29, 2020 (KR) 10-2020-0065172

(51) Int. Cl. G01N 1/40

(52) U.S. Cl. CPC G01N 1/4077; G01N 2001/4088

(57) ABSTRACT

Provided is a method for preparing cell-derived vesicles, and more particularly, a method for preparing cell-derived vesicles using a cell extruder, and a syringe-type cell extruder for effectively preparing cell-derived vesicles. According to the present invention, by using the method for preparing the cell-derived vesicles and the cell extruder, it is possible to prepare cell-derived vesicles in a stable, economical, and mass-producible manner.

### TECHNICAL FIELD

The present invention relates to a method for preparing cell-derived vesicles, and more particularly, to a method for preparing cell-derived vesicles using a cell extruder, and a syringe-type cell extruder for effectively preparing cell-derived vesicles.

### BACKGROUND ART

Recently, it has been reported that cell secretome contains various bioactive factors that control cell behavior, and particularly, the cell secretome contains ‘exosomes’ or ‘extracellular vesicles’, which are nanovesicles with an intercellular signaling function, and research on components and functions thereof has been actively conducted.

Cells release various membrane types of vesicles in an extracellular environment, and these released vesicles are commonly referred to as extracellular vesicles. The extracellular vesicles are also called cell membrane-derived vesicles, ectosomes, shedding vesicles, microparticles, exosomes, and the like, and in some cases, the extracellular vesicles are used separately from the exosomes.

The exosomes are vesicles with sizes of several tens to hundreds of nanometers composed of the same double phospholipid membrane as the structure of the cell membrane, and contain proteins, mRNAs, miRNAs, etc. called exosome cargo therein. The exosome cargo includes a wide range of signaling factors, and these signaling factors are known to be cell type-specific and differently regulated according to an environment of secretory cells. The exosomes are intercellular signaling mediators secreted from the cells, and it is known that various cellular signals transmitted through the exosomes regulate cell behavior, including activation, growth, migration, differentiation, dedifferentiation, apoptosis, and necrosis of target cells. The exosomes contain specific genetic materials and bioactive factors according to the nature and state of the derived cells. Proliferating stem cell-derived exosomes regulate cell behaviors such as migration, proliferation, and differentiation of the cells, and reflect the characteristics of stem cells related to tissue regeneration.

Conventional techniques for isolating such exosomes or extracellular vesicles include ultracentrifugation, density gradient centrifugation, ultrafiltration, size exclusion chromatography, ion exchange chromatography, immunoaffinity capture, microfluidics-based isolation, exosome precipitation, total exosome isolation kit, polymer based precipitation, or the like.

The ultracentrifugation is a method which has been so far the most widely used method to isolate the exosomes or extracellular vesicles, but has disadvantages of having low yield, requiring a lot of time to be isolated, being labor-intensive, and requiring expensive equipment. In addition, the ultracentrifugation has a disadvantage of damaging the exosomes or extracellular vesicles during the isolation process to interfere with subsequent analysis processes or applications.

The ultrafiltration may be used together with ultracentrifugation to increase the purity of exosomes or extracellular vesicles, but there is a problem that the exosomes or extracellular vesicles are attached to a filter to lower the yield after isolation.

In addition, the immunoaffinity capture has an advantage of high specificity as a method of isolating exosomes or extracellular vesicles by attaching the antibodies to the exosomes or extracellular vesicles, but there are disadvantages that a process of making the antibodies and a process of removing the antibodies after isolation are required, and the method is expensive, and the method is an unsuitable method for scale-up.

Meanwhile, recently, as a method of isolating exosomes, various exosome isolation kits such as exosome precipitation, total exosome isolation kit, or polymer-based precipitation are commercially sold. However, this method is easy to use, but the cost of reagents is high, so although the method may be used to isolate exosomes or extracellular vesicles at a laboratory level, there is a problem that the method is not suitable for isolating and purifying the exosomes or extracellular vesicles in large quantities.

Above all, in the isolation process of exosomes or extracellular vesicles, there are problems that the yield is low, a lot of time is required for the isolation and purification of exosomes or extracellular vesicles, and the method is cumbersome and expensive. In addition, there is a problem that conventional isolation methods developed to increase the purity make it difficult to scale-up and are unsuitable for Good Manufacturing Practice (GMP).

Therefore, in the technical field to which the present invention pertains, there is a steady demand for a technology capable of efficiently isolating and purifying the exosomes or extracellular vesicles.

### DISCLOSURE

### Technical Problem

The present inventors have studied a method of improving a process of extruding and preparing cell-derived vesicles, confirmed that the production efficiency of cell-derived vesicles varied depending on a ratio of the area of a penetrating portion through which a cell suspension passed to the area of a membrane filter, the number of intermediate filters, and an extrusion rate, confirmed that the extrusion efficiency was significantly increased at a predetermined area ratio, a predetermined number of intermediate filters, and a predetermined extrusion rate, and then completed the present invention.

Accordingly, an object of the present invention is to provide a method for efficiently preparing cell-derived vesicles.

### Technical Solution

One aspect of the present invention provides a method for preparing cell-derived vesicles including extruding a sample containing cells with a cell extruder including a supporter, an intermediate filter, and a membrane filter, in which the supporter supports the intermediate filter and the membrane filter, and includes a penetrating portion through which the sample containing the cells passes in the center of the supporter, and a ratio (S2/S1) of an area (S2) of the membrane filter to an area (S1) of the penetrating portion is 50 to 300.

Another aspect of the present invention provides a cell extruder including 1 to 10 intermediate filters; an O ring; a supporter having a penetrating portion formed in the center; an external casting; and a syringe, which are symmetrically connected to each other based on a membrane filter, in which a ratio (S2/S1) of an area (S2) of the membrane filter to an area (S1) of the penetrating portion of the supporter is 50 to 300.

### Advantageous Effects

According to the present invention, by using the method for preparing the cell-derived vesicles and the cell extruder, it is possible to prepare cell-derived vesicles in a stable, economical, and mass-producible manner.

### DESCRIPTION OF DRAWINGS

FIG. 1 is a diagram illustrating a syringe-type cell extruder.

FIG. 2 is a diagram illustrating an area of a region where a membrane filter is in contact with a supporter portion of an extruder and an area of a penetrating portion through which a cell suspension passes.

FIG. 3 is a diagram illustrating supporters and O rings used in experimental groups by varying an area of the penetrating portion and an area of the membrane filter.

FIG. 4 is a diagram illustrating a pressure applied to the membrane filter and an amount of the extruded sample when cells are extruded in experimental groups by varying an area of the penetrating portion and an area of the membrane filter.

FIG. 5 is a diagram illustrating a pressure applied to the extruder, a finally extruded cell suspension and the number of finally extruded cells, and a size of extruded cell-derived vesicles when varying the number of intermediate filters used during extrusion.

FIG. 6 is a schematic diagram illustrating a cell suspension diffusion effect according to the number of intermediate filters.

FIG. 7 is a diagram illustrating a size of cell-derived vesicles, a polydispersity index (PDI), and a concentration of cell-derived vesicles in an extrusion solution according to an extrusion rate.

### BEST MODE OF THE INVENTION

The present invention provides a method for preparing cell-derived vesicles including extruding a sample containing cells with a cell extruder including a supporter, an intermediate filter, and a membrane filter, in which the supporter supports the intermediate filter and the membrane filter, and includes a penetrating portion through which the sample containing the cells passes in the center of the supporter, and a ratio (S2/S1) of an area (S2) of the membrane filter to an area (S1) of the penetrating portion is 50 to 300.

As used herein, the term ‘cell-derived vesicles (CDVs)’ refer to vesicles artificially prepared from nucleated cells, and vesicles which are released from the cell membrane in almost all types of cells to have a double phospholipid membrane form which is the structure of the cell membrane. The cell-derived vesicles of the present invention may have a micrometer size, for example, 0.03 to 1 μm, and may be used interchangeably herein. The cell-derived vesicles of the present invention are distinguished from naturally secreted vesicles, and the ‘vesicles’ of the present invention have the inside and outside divided by a lipid double membrane consisting of cell membrane components of a derived cell, have cell membrane lipids, cell membrane proteins, nucleic acids, and cell components of the cell, and are smaller in size than an original cell, but are not limited thereto.

The “sample containing the cells” of the present invention may be a sample containing nucleated cells or transformed cells thereof, and may be a sample containing cells capable of preparing the vesicles without limitation.

In the present invention, the membrane filter may be used without limitation as long as the membrane filter has a filterable membrane structure having a pore size of 0.1 to 10 μm, preferably a polycarbonate membrane filter.

In the present invention, the intermediate filter may be a filter selected from the group consisting of polyester, nylon, polypropylene, polyurethane, acrylic fiber, vinylon, polyvinylidene chloride, polyvinyl acetate, wool, silk, cotton, hemp, and rayon, and preferably a filter made of a polyester material.

In the present invention, when the number of intermediate filters is varied and stacked, the production efficiency of cell-derived vesicles may be increased, which may be used in the same meaning as changing the total thickness of the stacked intermediate filters. In one embodiment, the intermediate filters having a thickness of 100 μm are stacked as a preferred embodiment, so that the total thickness of the stacked intermediate filters is 100 μm, 500 μm, or 1000 μm. If the single or stacked total thickness satisfies the thickness range of 10 μm to 1000 μm, the intermediate filter may be used without limitation so as to be single or stacked, and preferably 4 to 6 intermediate filters having a thickness of 90 to 110 μm may be stacked and used, or a single or stacked intermediate filters having a total thickness of 360 to 660 μm may be used.

In the present invention, the supporter serves to support the membrane filter and the intermediate filters, and any object having a structure including a penetrating portion through which the cell suspension passes in the center of the supporter may be used without limitation.

In the present invention, the ratio (S2/S1) of the area (S2) of the membrane filter to the area (S1) of the penetrating portion may be 50 to 300, preferably the ratio (S2/S1) may be 70 to 170.

When the cell-derived vesicles are prepared with a cell extruder to which the ratio (S2/S1) of the present invention is applied, the extrusion stability is increased because an excessive load is not applied to the membrane filter during extrusion, and the amount of extruded sample is increased to increase the extrusion efficiency for preparing the cell-derived vesicles.

In the present invention, when the ratio (S2/S1) is 70 to 170, 4 to 6 intermediate filters having a thickness of 90 μm to 110 μm may be stacked and used, and the intermediate filters to be single or stacked may be used so that the total thickness thereof is 360 μm to 660 μm.

When the cell-derived vesicles are prepared with the cell extruder to which the thickness and number of intermediate filters of the present invention are applied, an effect of pressure reduction caused when the sample including cells reaches the membrane filter in a large area is greater than the resistance received while passing through the intermediate filters, and thus, the load of the membrane filter is lowered, thereby increasing the extrusion efficiency for preparing the cell-derived vesicles.

In the present invention, the extruding may be performed at an extrusion rate of 10 to 90 ml/min, preferably at an extrusion rate of 60 to 90 ml/min. When extruded at an extrusion rate of about 60 ml/min, the yield of cell-derived vesicles may be the highest, and when the extruding is performed at 60 ml/min or more, the PDI of the extruded sample may be 0.3 or less, which means that the sizes of the extruded cell-derived vesicles are constant, and thus, the utilization of the cell-derived vesicles is excellent.

When the cell-derived vesicles are prepared by applying the extrusion rate of the present invention, it may be possible to prepare the cell-derived vesicles with high yield and high quality.

By applying all of the ratio (S2/S1), the thickness and number of the intermediate filters, and the extrusion rate of the present invention, it is possible to prepare cell-derived vesicles. In this case, since stable cell extrusion is possible, it may be possible to extrude a large amount of samples and it may be possible to prepare the cell-derived vesicles with high yield and high quality.

In addition, the present invention provides a method for preparing cell-derived vesicles including extruding a sample containing cells with a cell extruder including a supporter, an intermediate filter, and a membrane filter at an extrusion rate of 60 to 90 ml/min.

In the present invention, when preparing the cell-derived vesicles, including extruding the cell-derived vesicles at the extrusion rate of 60 to 90 ml/min, the yield of the cell-derived vesicles may be the highest. When the extruding step is performed at 60 ml/min or more, the polydispersity index (PDI) of the extruded sample may be or less, which means that the sizes of the extruded cell-derived vesicles are constant, and thus, the utilization of the cell-derived vesicles is excellent.

In addition, the present invention provides a method for preparing cell-derived vesicles including extruding a sample containing cells with a cell extruder including a supporter, an intermediate filter, and a membrane filter, in which the intermediate filter has a total thickness of 360 to 660 μm.

In the present invention, when the cell-derived vesicles are prepared with the cell extruder in which the total thickness of the intermediate filters is 360 to 660 μm, an effect of pressure reduction caused when the sample including the cells reaches the membrane filter in a large area is greater than the resistance received while passing through the intermediate filters, and thus, the load of the membrane filter is lowered, thereby increasing the extrusion efficiency for preparing the cell-derived vesicles.

Further, the present invention provides a syringe-type cell extruder in which intermediate filters, O-rings, supporters, external castings, and syringes are sequentially and/or symmetrically connected to each other based on a membrane filter.

Hereinafter, the cell extruder of the present invention will be described in detail with reference to the accompanying drawings.

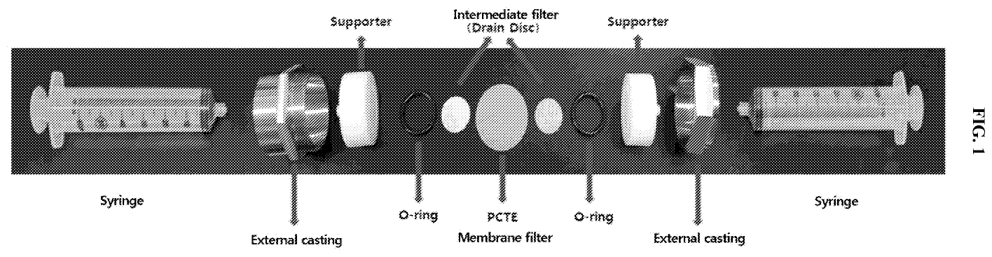

FIG. 1 is an exploded part diagram of a cell extruder according to the present invention, and the cell extruder may be completed by recombining the parts in the illustrated order.

As illustrated in FIG. 1, in the cell extruder according to the present invention, intermediate filters, O rings fixing the membrane filter and the intermediate filters, supporters including penetrating portions through which the sample containing the cells pass in the center, external castings that block the rest of components except for the syringes from the outside, and syringes by which the sample including the cells is dispensed are sequentially and/or symmetrically configured based on the membrane filter.

In the present invention, the ratio (S2/S1) of the area (S2) of the membrane filter to the area (S1) of the penetrating portion may be 50 to 300, preferably the ratio (S2/S1) may be 70 to 170.

When the cell-derived vesicles are prepared with the cell extruder of the present invention to which the ratio (S2/S1) of the present invention is applied, since an excessive load is not applied to the membrane filter during extrusion, the extrusion stability is increased, and the amount of the extruded sample is increased, thereby increasing the extrusion efficiency for preparing the cell-derived vesicles.

In the present invention, the intermediate filter may be configured by stacking 4 to 6 intermediate filters having a thickness of 90 μm to 110 μm, or the intermediate filter may be configured singly or by stacking the intermediate filters so that the total thickness of the single or stacked intermediate filters is 360 μm to 660 μm.

When the cell-derived vesicles are prepared with the cell extruder to which the thickness and number of intermediate filters of the present invention are applied, an effect of pressure reduction caused when the sample including cells reaches the membrane filter in a large area is greater than the resistance received while passing through the intermediate filters, and thus, the load of the membrane filter is lowered, thereby increasing the extrusion efficiency for preparing the cell-derived vesicles.

In the present invention, the intermediate filter may be configured by stacking 4 to 6 intermediate filters having a thickness of 90 μm to 110 μm, or the intermediate filter may be configured singly or by stacking the intermediate filters so that the total thickness of the single or stacked intermediate filters is 360 μm to 660 μm. In addition, the ratio (S2/S1) of the area (S2) of the membrane filter to the area (S1) of the penetrating portion may be 50 to 300, preferably the ratio (S2/S1) may be 70 to 170.

When the cell-derived vesicles are prepared with the cell extruder to which the thickness and number of intermediate filters of the present invention and the ratio (S2/S1) are applied, excessive load is not applied to the membrane filter during extrusion, and an effect of pressure reduction caused when the sample including cells reaches the membrane filter in a large area is greater than the resistance received while passing through the intermediate filters, and thus, the load of the membrane filter is lowered, thereby increasing the extrusion efficiency for preparing the cell-derived vesicles.

### MODES OF THE INVENTION

### Example 1. Comparison of Cell Extrusion Efficiency According to Ratio of

Area of Penetrating Portion of Supporter and Area of Membrane Filter 1.1. Fabrication of Cell Extruder

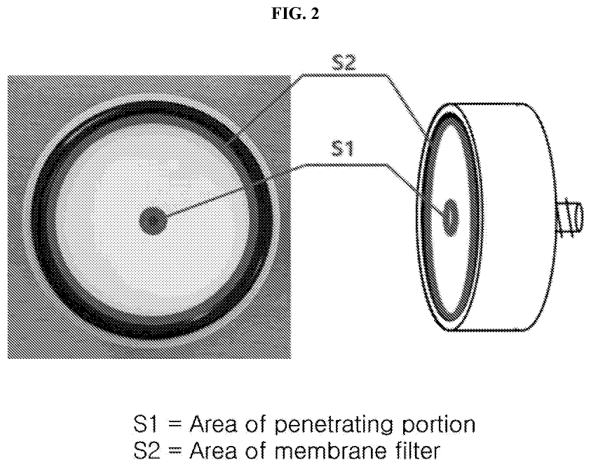

A cell extruder for preparing extracellular vesicles by extruding cells was fabricated in the form of a syringe-type extruder equipped with a syringe pump. As the membrane filter, a Whatman PC membrane filter 5 μm with a pore size of 5 μm was used. An intermediate filter (drain disc) is a disc with a flat surface, used to prevent rupture of the membrane filter, and is made of a chemically inert polyester material with a thickness of 100 μm. In order to prevent the excessive load from being applied to the membrane filter, a syringe pump is designed to be stopped when a pressure of 50 psi or more is applied to the syringe pump. The fabricated syringe-type extruder is illustrated in FIG. 1, and the supporter in the extruder is illustrated in FIG. 2.

1.2. Evaluation of Extrusion Stability According to Ratio of Area of Membrane Filter and Area of Penetrating Portion of Supporter

When a ratio of an area of the membrane filter and an area of the penetrating portion in the supporter was varied, an experiment was performed to confirm the cell extrusion efficiency.

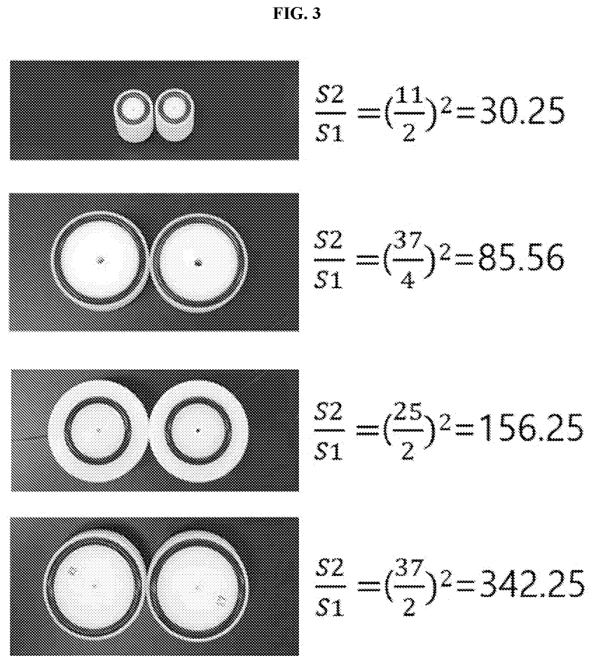

As an experimental group, four experimental groups were set by varying an area (S1) of the penetration portion and an area (S2) of the membrane filter, and were illustrated in Table 1. The supporters fabricated according to the set experimental groups were illustrated in FIG. 3.

TABLE 1

| | S1 (area of | S2 (area of membrane | |
|---|---|---|---|
| | penetrating portion) | filter) | S2/S1 |
| Experimental | π mm² (radius: 1 | 30.25π mm² (radius 5.5 | 30.25 |
| Group 1 | mm) | mm) | |
| Experimental | 4π mm² (radius: 2 | 342.25π mm² (radius 18.5 | 85.56 |
| Group 2 | mm) | mm) | |
| Experimental | π mm² (radius: 1 | 156.25π mm² (radius 12.5 | 156.25 |
| Group 3 | mm) | mm) | |
| Experimental | π mm² (radius: 1 | 342.25π mm² (radius 18.5 | 342.25 |
| Group 4 | mm) | mm) | |

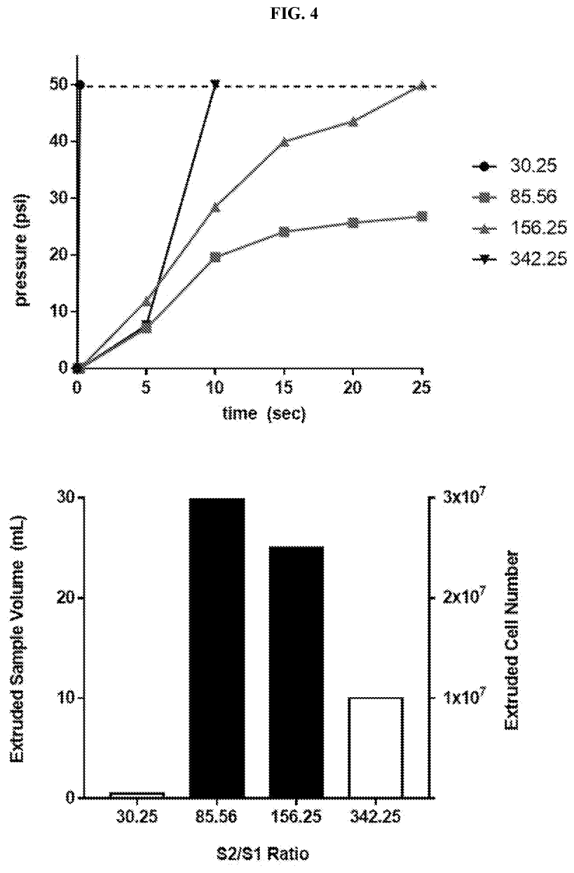

Extrusion efficiency was measured according to the set area ratio (S2/S1) of the penetrating portion and the membrane filter. Specifically, 30 mL of a cell suspension obtained by diluting human macrophage cell line U937 cells with a cell size of 9.8 μm in a phosphate buffer at a concentration of 1×10⁶ cells/mL was extruded at a rate of 60 ml/min with the extruder fabricated in Example 1.1 by varying the area of the penetrating portion in the supporter and the area of the membrane filter and setting the number of intermediate filters to 5 like the set experimental group. The pressure applied to the extruder, the finally extruded cell suspension, and the number of extruded cells were measured, and the results were illustrated in FIG. 4. As illustrated in FIG. 4, in the case of Experimental Group 1, the pressure applied to the membrane filter immediately after the starting of extrusion exceeded 50 psi, so that the extrusion was hardly performed. In the case of Experimental Group 2, it was confirmed that the pressure applied to the membrane filter did not exceed 30 psi even after 25 seconds had elapsed from the start of extrusion, so that stable extrusion was possible and the entire cell suspension was extruded. In the case of Experimental Group 3, after about 25 seconds had elapsed from the start of extrusion, the pressure applied to the membrane filter exceeded 50 psi, and accordingly, it was confirmed that about 25 ml of 30 ml of the cell suspension was extruded. In the case of Experimental Group 4, after about 10 seconds had elapsed from the start of extrusion, the pressure applied to the membrane filter exceeded 50 psi, and accordingly, it was confirmed that about 10 ml of 30 ml of the cell suspension was extruded, but the syringe pump was stopped.

Through these results, it was confirmed that when the S2/S1 value was within the range of 50 to 300, efficient extrusion was possible and the extrudable amount was significantly increased. When the S2/S1 value is less than 50, the area of the membrane filter was absolutely insufficient to extrude a large volume of cell suspension, and a pressure difference before and after the membrane rose sharply to reach a pressure limit line of the syringe pump, and then the extrusion did not proceed. When the S2/S1 value exceeded 300, it was confirmed that the time for the pressure difference before and after the membrane to reach the pressure limit line was shortened to cause the stopping of the syringe pump, and the extrudable amount decreased. In the process shown in the data, 9.8 μm of U937 cells were extruded through a membrane filter with a pore size of 5 μm, and as a result, it was confirmed that cell vesicles with an average diameter of 400 to 600 nm were produced.

### Example 2. Comparison of Extrusion Efficiency According to Number of Intermediate Filters

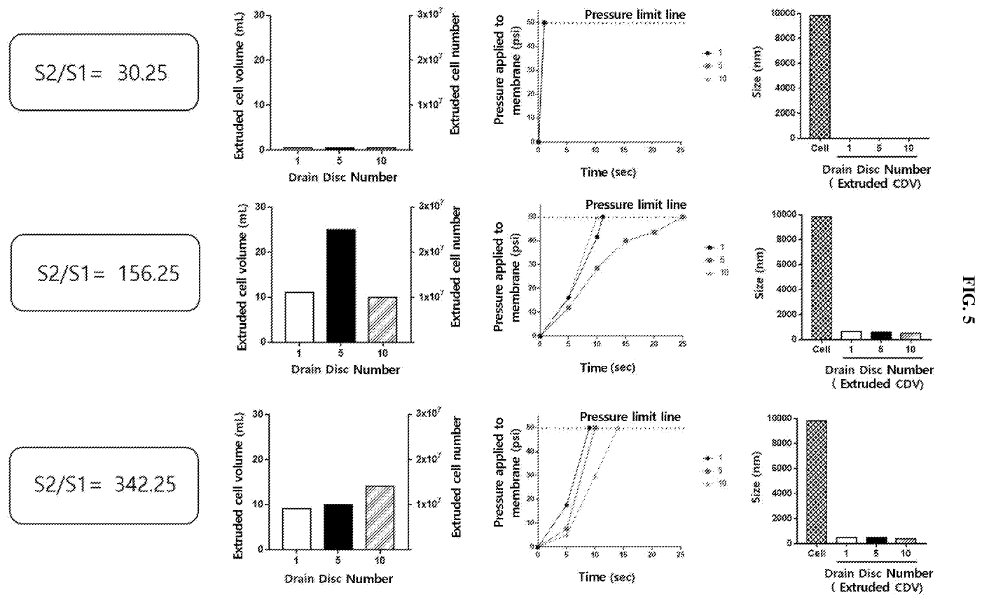

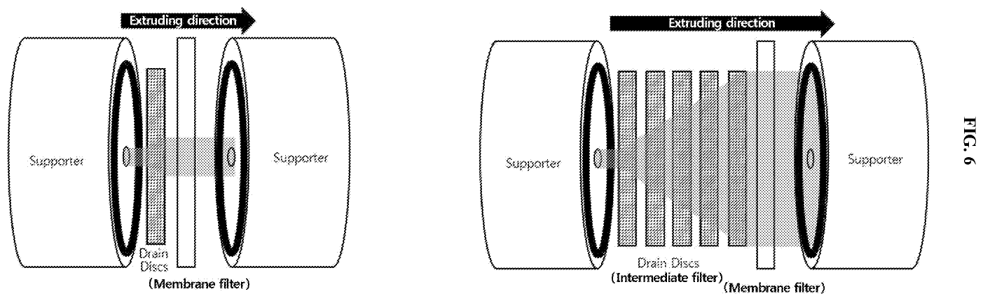

When the number of intermediate filters used during cell extrusion was varied, an experiment was performed to evaluate cell extrusion efficiency. Specifically, in the experimental groups in which the area ratios (S2/S1) of the penetrating portion and the membrane filter were set to 30.25, 156.25, and 342.25, the extruding was performed in the extruder fabricated in Example 1.1 at a rate of 60 ml/min by varying the number of intermediate filters to 1, 5, and 10, respectively, and a pressure applied to the extruder, a volume of the finally extruded cell suspension, the number of extruded cells, and a size of the extruded cell-derived vesicles were measured, and these results were illustrated in FIG. 5. In addition, a schematic diagram illustrating a cell suspension diffusion effect according to the number of intermediate filters was illustrated in FIG. 6.

As illustrated in FIG. 5, when 5 intermediate filters were connected to the front of the membrane filter, the amount of extrusion during extruding was significantly increased compared to 1 or 10 intermediate filters. As a result, it was confirmed that the number of extruded cells was increased, and the number of obtained cell-derived vesicles was also significantly increased. In Experimental Group with the S2/S1 value of 156.25, when 5 intermediate filters were connected, it took about 25 seconds for the pressure to rise to the pressure limit line (50 psi), and thus, it was confirmed that the pressure was increased slowly to a significant extent compared to a case where 1 or 10 intermediate filters were connected, and as a result, the operating time was increased, and then the amount of extrusion was increased. As a result, when one intermediate filter was connected and extruded, since the diffusion range of the cell suspension passing through the intermediate filter was decreased, the extrusion efficiency decreased as the area of the membrane filter reached by the cell suspension decreased. However, when 10 or more intermediate filters were connected, it was confirmed that the resistance received while the cell suspension passed through the intermediate filters became greater than the effect of pressure reduction due to the increase in area due to diffusion, so that the extrusion efficiency decreased.

In addition, as a result of measuring the size of the cell-derived vesicles produced in Example 2, it was confirmed that the cells having a diameter of 9800 nm were extruded to the cell-derived vesicles of about 400 to 600 nm through an extruding process through a membrane filter having pores of 5 μm.

These results show that the extrusion efficiency of the cell-derived vesicles may be increased by controlling the number of intermediate filters, that is, the total thickness of the intermediate filters, in addition to the ratio (S2/S1) of the area of the membrane filter to the area of the penetrating portion in the supporter.

### Example 3. Comparison of Extrusion Efficiency According to Extrusion Rate

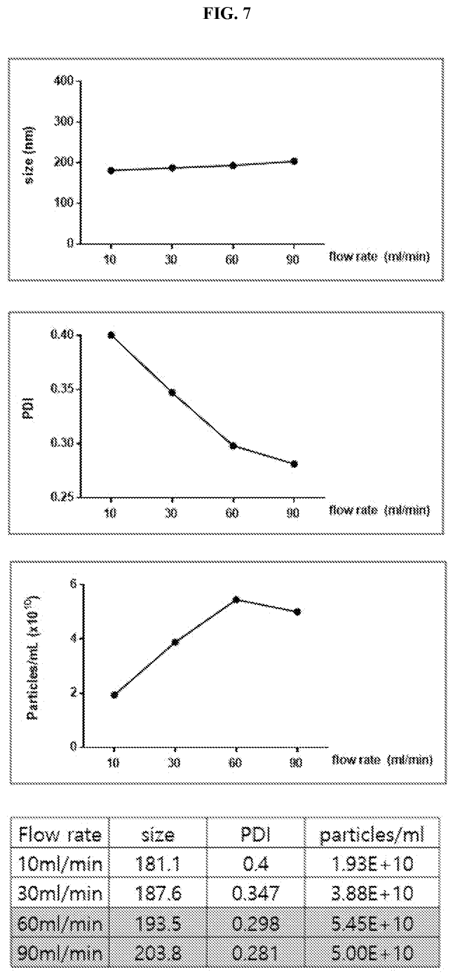

In order to vary the extrusion rate during cell extrusion, experimental groups were set by setting the movement rate of the syringe pump to 10 ml/min, 30 ml/min, ml/min, and 90 ml/min, respectively, and the extruding was performed in the cell extruder including the supporters, the intermediate filters, and the membrane filter, in which the S2/S1 value was 156.25 and the number of intermediate filters was 5. The sizes of cell-derived vesicles according to the extrusion rate, polydispersity indexes (PDI), and the concentrations of cell vesicles in the extrusion solution were compared, and the results were illustrated in FIG. 7.

As illustrated in FIG. 7, it was confirmed that the size of the extruded cell-derived vesicles in each experimental group was measured to be about 180 to 210 nm, and the average diameter of the cell-derived vesicles was similar even if the extrusion rate was changed. It was confirmed that the higher the extrusion rate, the lower the PDI was measured, and the sizes of the cell-derived vesicles became uniform when the extrusion rate was high. In addition, when extruded at a rate of 60 ml/min or more, it was confirmed that the production rate was improved by increasing the amount of cell-derived vesicles in the extrusion solution, and particularly, when extruded at 60 ml/min, it was confirmed that the concentration of cell-derived vesicles in the extrusion solution was 5.45×10¹⁰/ml, indicating the highest production rate.

As described above, specific parts of the present invention have been described in detail, and it will be apparent to those skilled in the art that these specific techniques are merely preferred embodiments, and the scope of the present invention is not limited thereto. Therefore, the substantial scope of the present invention will be defined by the appended claims and their equivalents.

1. A method for preparing cell-derived vesicles comprising extruding a sample containing cells with a cell extruder including a supporter, an intermediate filter, and a membrane filter, wherein the supporter supports the intermediate filter and the membrane filter, and includes a penetrating portion through which the sample containing the cells passes in a center of the supporter, and a ratio (S2/S1) of an area (S2) of the membrane filter to an area (S1) of the penetrating portion is 50 to 300.

2. The method for preparing the cell-derived vesicles of claim 1, wherein the membrane filter has a pore size of 0.1 to 10 μm.

3. The method for preparing the cell-derived vesicles of claim 1, wherein the ratio (S2/S1) of the area (S2) of the membrane filter to the area (S1) of the penetrating portion is 70 to 170.

4. The method for preparing the cell-derived vesicles of claim 1, wherein the intermediate filter has a thickness of 90 to 110 μm, and 4 to 6 intermediate filters are stacked.

5. The method for preparing the cell-derived vesicles of claim 1, wherein the intermediate filter has a total thickness of 360 to 660 μm.

6. The method for preparing the cell-derived vesicles of claim 1, wherein the extruding is performed at an extrusion rate of 60 to 90 ml/min.

7. (canceled)

8. (canceled)

9. A cell extruder having a syringe type comprising:
1) an intermediate filter;
2) an O-ring;
3) a supporter having a penetrating portion in a center;
4) an external casting; and
5) a syringe, which are sequentially connected to each other based on a membrane filter, wherein a ratio (S2/S1) of an area (S2) of the membrane filter to an area (S1) of the penetrating portion of the supporter is 50 to 300.

10. The cell extruder of claim 9, wherein the ratio (S2/S1) of the area (S2) of the membrane filter to the area (S1) of the penetrating portion of the supporter is 70 to 170, the intermediate filter has a thickness of 90 to 110 μm, and 4 to 6 intermediate filters are stacked.

11. The cell extruder of claim 9, wherein the ratio (S2/S1) of the area (S2) of the membrane filter to the area (S1) of the penetrating portion of the supporter is 70 to 170, and the intermediate filter has a total thickness of 360 to 660 μm.

12. (canceled)

13. A cell extruder having a syringe type comprising:
1) an intermediate filter;
2) an O-ring;
3) a supporter having a penetrating portion in a center;
4) an external casting; and
5) a syringe, which are sequentially connected to each other based on a membrane filter, wherein the intermediate filter has a total thickness of 360 to 660 μm.
